(19) United States
(12) Patent Application Publication
(10) Pub. No.: US 2007/0276142 A1
(43) Pub. Date: Nov. 29, 2007

(54) Catalytic Asymmetric Synthesis of Optically Active Alpha-Halo-Carbonyl Compounds

(76) Inventors: Nis Halland, Bad Soden (DE); Karl Jorgensen, Arhus (DK); Mauro Marigo, Tilst (DK); Alan Braunton, Harlow (GB); Stephan Bachmann, Allschwil (CH); Doris Schluter, Darmstadt (DE)

Correspondence Address: JACOBSON HOLMAN PLLC, 400 SEVENTH STREET N.W., SUITE 600, WASHINGTON, DC, 20004, US

(21) Appl. No.: 10/589,256

(22) PCT Filed: Feb. 11, 2005
(86) PCT No.: PCT/DK05/00094

§ 371 Date: Jun. 25, 2007

(30) Foreign Application Priority Data
Feb. 19, 2004 (DK) PA 2004 00252
Jun. 1, 2004 (DK) PA 2004 00857
Dec. 20, 2004 (DK) PA 2004 01960
Jan. 11, 2005 (DK) PA 20005 00047

(51) Int. Cl. C07D 207/04; C07C 45/00; C07D 211/00; C07D 211/70; C07D 317/72

(52) U.S. Cl. 546/242; 548/570; 548/572; 548/578; 549/342; 568/348; 568/393

(57) ABSTRACT

A process for the catalytic asymmetric synthesis of an optically active compound of the formula (1a) or (1b) 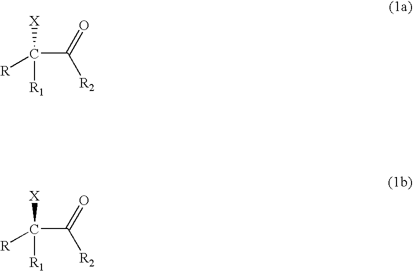
wherein R is an organic group; X is halogen; R₁ and R₂ which may the same or different represents H, or an organic group or R₁ and R₂ may be bridged together forming part of a ring system; R and R₂ may be bridged together forming part of a ring system; with the provisio that R and R₁ are different and R₂, when different from H, is attached though a carbon-carbon bond,
comprising the step of reacting a compound of the formula (2) 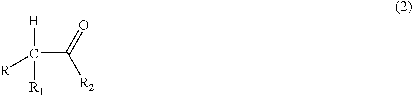 with a halogenation agent in the presence of a catalytic amount of a chiral nitrogen containing organic compound.

### BACKGROUND

The present invention is related to a process for the catalytic asymmetric synthesis of optically active α-halo-carbonyl compounds of the formula (1) 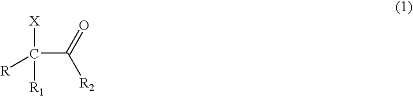
wherein R is an organic group; X is halogen; R₁ and R₂ which may be the same or different represents H, or an organic group, or R₁ and R₂ may be bridged together forming part of a ring system; R and R₂ may be bridged together forming part of a ring system; with the provisio that R and R₁ are different and R₂ when different from H is attached through a carbon-carbon bond.

An important goal for asymmetric catalysis is to develop new reactions affording optically active building blocks using simple and easily-available starting materials and catalysts. Optically active halogen containing compounds are especially attractive due to their high value as synthetic intermediates. Despite intensive research efforts over the past years, examples of highly enantioselective halogenation reactions are scarce and often limited to 1,3-dicarbonyl compounds or multi-step procedures requiring expensive reagents.

The compounds of general formula (1) are e.g. useful intermediates for the syntheses of pharmaceuticals such as antibiotics, agrochemicals, raw materials for chemicals and the like.

### DESCRIPTION OF THE INVENTION

In a first embodiment, the present invention provides a one-step catalytic asymmetric process for the synthesis of an optically active compound of formula (1a) or (1b) 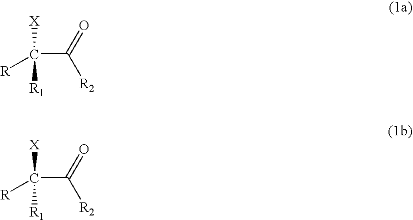
wherein R is an organic group; X is halogen; R₁ and R₂ which may be the same or different represents H or an organic group, or R₁ and R₂ may be bridged together forming part of a ring system; R and R₂ may be bridged together forming part of a ring system; with the provisio that R and R₁ are different and R₂ when different from H is attached through a carbon-carbon bond and,
comprising the step of reacting a compound of the formula (2) 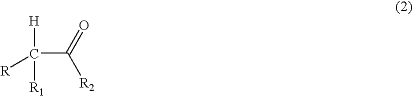
with a halogenating agent and in the presence of a catalytic amount of a chiral nitrogen containing organic compound.

The compound represented by the general formula (1) is not limited to specified ones, as long as the object of the present invention is not hindered. In the general formula (1), R, R₁, R₂ includes, for instance, alkyl groups, allenyl groups, allkynyl groups, haloallcyl groups, alkylaryl groups, aryl groups and heterocyclic groups, each of which may have one or more substituents.

For convenience, certain terms employed in the specification, examples and claims are collected here.

The term “catalytic amount” is recognized in the art and means a sub-stoichiometric amount relative to a reactant. As used herein, a catalytic amount means from 0.0001 to 90 mole percent relative to a reactant, preferably from 0.001 to 50 mole percent, and more preferably from 0.1 to 20 mole percent relative to a reactant.

The term “enantiomeric excess” (ee) is well known in the art and is defined for a resolution of the racemic mixture
ab→a+b as

$${ee}_{a} = ( \frac{conc . of a - conc . of b}{conc . of a + conc . of b} ) \times 100$$

The value of ee will be a number between 0 and 100, zero being racemic and 100 being pure single enantiomer.

The term “alkyl” refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. Moreover, the term alkyl as used throughout the specification and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a hydroxyl, a carbonyl, an alkoxyl, an ester, a phosphoryl, an amine, an amide, an imine, a silyl, a silyl ether, a thiol, a thioether, a thioester, a sulfoxide, a sulfonyl, an amino, a nitro, a phosphino, a phosphate, an aryl, a heterocycle or an organometallic moiety. Representative examples of the alkyl group include groups having 1 to 20 carbon atoms in its hydrocarbon backbone, preferably 1 to 10 carbon atoms. When appropriate the number of carbon atoms designated in the hydrocarbon backbone for a substituent is assigned (i.e. C₁₋₇ means one to seven carbons). It will be understood by those skilled in the art that the moieties substituted on the hydrocarbon chain can themselves be substituted, if appropriate.

The term “alkenyl” refers to linear or branched groups of 2 to about 20 carbon atoms or, preferably, 2 to about 8 carbon atoms, having at least one carbon-carbon double bond. The term is intended to include both “unsubstituted alkenyls” and “substituted alkenyls” as described for alkyl above.

The term “alkynyl” refers to linear or branched groups of 2 to about 20 carbon atoms or, preferably, 2 to about 8 carbon atoms, having at least one carbon-carbon triple bond. The term is intended to include both “unsubstituted alkynyls” and “substituted alkynyls” as described for alkyl above.

The term “haloalkyl” refers to an alkyl group, as defined above, wherein one or more hydrogen atoms are replaced by a halogen atom.

The term “aryl” refers to a carbocyclic aromatic system containing one or more rings wherein such rings may be attached together in a pendent manner or may be fused. Examples of aryl groups include phenyl, naphthyl, tetrahydronaphthyl, indane and biphenyl. The aromatic ring can be substituted at one or more ring positions with such substituents as described above, as for example, halogens, alkyls, haloalkyls, alkenyls, alkynyls, hydroxyl, amino, nitro, thiol, amines, imines, amides, carbonyls, carboxyls, ethers, thioethers, sulfonyls, sulfoxides, phosphinos, phosphonates, ketones, aldehydes, esters or the like.

The term “alkylaryl” refers to aryl-substituted alkyl groups. Preferable alkylaryl groups are “lower alkylaryl” groups having aryl groups attached to alkyl groups having 1 to 6 carbon atoms. Even more preferred lower alkylaryl groups are phenyl attached to alkyl portions having 1 to 3 carbon atoms. Examples of such groups include benzyl, diphenylmethyl and phenylethyl. The aryl in said alkylaryl may be additionally substituted as defined above. When appropriate the number of carbon atoms designated in the hydrocarbon backbone of the alkyl part is assigned (i.e. C₁₋₃ alklylaryl means an alkylaryl group where the alkyl part contains one to three carbon atoms).

The term “heterocyclic” refers to 3 to 10-membered ring structures, which include at least one heteroatom preferably selected from O, S or N, and which may be aromatic (heteroaryl). Examples of such structures include pyridine, pyrimidine, piperidine, triazole, thiophene, furane, morpholine, chromane, indole, oxazole etc. The heterocycle may be substituted in one or more ring positions as mentioned for the aryl groups.

The term “amino” refers to a primary, secondary or tertiary amino group bonded via the nitrogen atom, with the secondary amino group carrying an alkyl or phenyl substituent and the tertiary amino group carrying two similar or different substituents or the two nitrogen substituents together forming a ring. The substituents may be additionally substituted as defined above, and as such the amino group may form part of an amino acid moiety.

The tenn “silyl” refers to the -SiZ₁Z₂Z₃ group, where each of Z₁, Z₂ and Z₃ is independently selected from the group consisting of hydrogen and optionally substituted alkyl, alkenyl, alkynyl, aryl, alkylaryl, heterocyclic, alkoxy and amino.

The term “phosphino” refers to the group -PZ₁Z₂, where each of Z₁ and Z₂ is independently selected from the group consisting of hydrogen and optionally substituted alkyl, alkenyl, alkynyl, aryl, alkylaryl, heterocyclic and amino.

The term “phosphate” refers to the group —O(P═O)(OZ₁)(OZ₂) where Z₁ and Z₂ is independently selected from the group consisting of hydrogen and optionally substituted alkyl and aryl,

The term “thio” is used herein to refer to the group —SZ₁, where Z, is selected from the group consisting of hydrogen and optionally substituted alkyl, alkenyl, alkynyl, aryl, alkylaryl and heterocyclic.

The term “sulfoxide” refers to the group —S(═O)Z₁ where Z₁ is selected from the group consisting of optionally substituted alkyl and alkylaryl.

The term “sulfonyl” refers to the group —SO₂Z₁ where Z₁ is selected from the group consisting of optionally substituted alkyl and alkylaryl.

When two substituents are bridged together, they are joined through a bridging group, e.g. via an alkylene, alkenylene, or alkynylene radical chain optionally with one or more of the carbon atoms substituted with a heteroatom, said chain optionally being substituted with one or more substituents.

The term “halogen” designates F, Cl, Br or I.

When any variable may occur more than one time in any formula for a compound, its definition on each occurrence is independent of its definition at every other occurrence.

R is preferably an optionally substituted C₁₋₁₀ alkyl group, an optionally substituted C₂₋₈ allylene group or a C₁₋₃-alkylaryl group. More preferably R is an optionally substituted C₁₋₆ alkyl group, an optionally substituted C₂₋₄ alkylene group or a C₁₋₂-alkylaryl group.

R₁ is preferably H or an optionally substituted C-₁₋₁₀ alkyl group.

R₂ is preferably H or an optionally substituted C₁₋₁₀ alkyl group or R and R₂ are bridged together forming part of a ring system. More preferably R₂ is H or together with R forms an optionally substituted C₃₋₅-alkylene bridge.

X is preferably F, Cl or Br.

In a preferred embodiment of the present invention R₁ and R₂ both represents H and R represents an optionally substituted C₁₋₁₀ alkyl group, an optionally substituted C₂₋₄ alkylene group or a C₁₋₂-alkylaryl group. More preferably R is attached through a —CH₂— group.

In another preferred embodiment of the present invention R₁ is H and R and R₂ each represents an optionally substituted C₁₋₁₀ alkyl group or R₂ together with R forms an optionally substituted C₃₋₅-alkylene bridge optionally with one or more of the carbon atoms being replaced by a heteroatom.

In principle any solvent that is capable of dissolving the reagents and the catalysts in suitable amounts and which is inert with respect of the reaction may be used. The solvent employed in the reaction may be either protic, aprotic, mixtures of both or ionic liquids. Suitable protic solvents include, water, alcohols e.g. straight, branched or cyclic allkanols and halogenated alkanols, aromatic alcohols; amines and organic acids. Suitable aprotic solvents include dioxane, tetrahydrofuran (THF), dimethylformamide (DMF), N-methylpyrrolidone, dimethylsulfoxide (DMSO), pyridine, alkanes and haloallkanes, ethers, ketones, aldehydes, nitriles, and nitroalkanes. The compound of formula (2) may also serve the purpose of solvent when in its liquid state at the reaction temperature.

Examples of halogenating agents are: N-halogenated amides such as, N-halosuccinimides e.g. N-chlorosuccinimide, N-bromosuccinimide or N-iodosuccinimide, N-halophthalimide e.g. N-chlorophthalimide, N,N′-dihalodimethylhydantoin e.g. N,N′-dichlorodimethylhydantoin, N-halosaccharine e.g. N-chlorosaccharine or N-bromosaccharine, 1,3,5-trihalo-1,3,5-triazine-2,4,6-trione e.g. 1,3,5-trichloro-1,3,5-triazine-2,4,6-trione, N-haloglutarimide e.g. N-chloroglutarimide, N-chloro-N-cyclohexyl-benzenesulfonimide; interhalogen compounds such as ICl or IBr; SO₂X₂ e.g. SO₂Cl₂; (Ph)₃PX₂ e.g. (Ph)₃PCl₂ or (Ph)₃PBr₂; (Ph)₃/CX₄ e.g. [(Ph)₃CCl₃]Cl; complexed halogens such as pyridin-HBr-Br₂ or (CH₃)₂S-Br₂; t-BuOCl; elemental halogen e.g. Cl₂ or Br₂; 2,3,4,5,6,6-hexachloro-2,4-cyclohexadien-1-one; 2,4,4,6-tetrabromo-2,5-cyclohexadien-1-one; 4,4-dibromo-2,6-di-tert-butyl-cyclohexa-2,5-dienone and electrophilic fluorinating agents such as N-fluorodibenzenesulfonimide (NFSI), 1-chloromethyl-4-fluoro- 1,4-diazoniabicyclo[2.2.2]octane bis-(tetrafluoroborate) (Selectflour®) and 1-methyl-4-fluoro-1,4-diazoniabicyclo[2.2.2]octane bis-(tetrafluoroborate).

Preferred halogenating agents are N-chlorosuccinimide (NCS), N-bromosuccinimide (NBS), 4,4-dibromo-2,6-di-tert-butyl-cyclohexa-2,5-dienone and N-fluorodibenzenesulfonimide (NFSI).

The amount of halogenating agent relative to the compound (2) depends on the amount of ‘active’ haloatoms on the halogenating agent, but in case of one active haloatom as in N-halosuccinimide, the amount is usually 0.25-4 equivalents, preferably 0.25-2.5.

It has further been found that addition of acids to the reaction media has a positive effect on the reaction rate and yield of the compound (1). Preferably the acid(s) is selected among carboxylic acids such as aliphatic and aromatic carboxylic acids. Examples of such acids are acetic acid, trifluoroacetic acid, chloroacetic acid, benzoic acid and nitro substituted benzoic acids e.g. 2-nitrobenzoic acid. The amount of acid relative to the compound (2) is 0-200 mole percent, preferably 0-60 mole percent.

Any chiral nitrogen containing organic compound capable of inducing asymnmetric halogenation can be used as catalyst. Preferred are catalysts having a primary or secondary nitrogen atom. It is to be understood that the chiral nitrogen containing organic compound may be used as such or when appropriate in one of its salt forms.

Examples of the chiral nitrogen containing organic compound used as catalyst include, but are not limited to, the following compound (3): 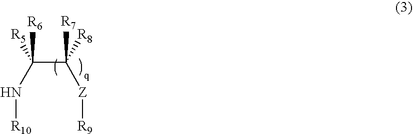
wherein q is 0 or 1;
R₅, R₆, R₇, R₈, which may be the same or different represents H, alkyl, haloalkyl, alkoxyl, OH, amino, amide, silyl, silyl ether, COR₁₁, optionally substituted aryl, an optionally substituted heterocycle, alkyl substituted with at least one OH group, an optionally substituted amino group or optionally substituted aryl or heterocycle or R₅ and R₆ together or R₇ and R₈ together may represent a carbonyl group or when q is 1, R₅ with either R₇ or R₈ may be bridged together forming part of a ring system; R₁₁ represents an optionally substituted amino group or OR₁₂ wherein R₁₂ represents H, alkyl or phenyl;
R₉ and R₁₀, which may the same or different represents H, alkyl, OH, alkoxy or R₉ and R₁₀ may be bridged together forming part of a ring system;
Z is S, O, C═O, C(R₁₄)₂, N—R₁₄ wherein R₁₄ is R₅;
with the provisio that the groups R₅, R₆, R₇, R₈, R₉, R₁₀, R₁₄, and Z are selected so that the compound (3) is a chiral compound.

It is within the capabilities of the skilled person to select suitable groups R₅, R₆, R₇, R₈, R₉, R₁₀, R₁₄, and Z so that the compound (3) will be a chiral compound. It will be immediately apparent for the skilled person which limitation this provisio provides to the selection. For example if q is 0 then may R₅ and R₆ be selected so that R₅ is different from R₆ and if q is 1 the may R₅, R₆, R₇ and R₈ be selected so that at least one of R₅, R₆, R₇ and R₈ is different from the three other of these.

In a preferred embodiment of the present invention, q is 1; R₅, R₆, R₇, R₈ which may be the same or different represents H, COR₁₁, optionally substituted aryl preferably phenyl or benzyl, or methyl substituted with at least one of the following, an OH group, an optionally substituted amino group or an optionally substituted aryl or heterocycle group; or R₅ and R₇ together represents a C₃₋₅ alkylene bridge;

R₁₁ represents OH, NH2 or NH-alkyl;

R₉ and R₁₀ are H or R₉ and R₁₀ together represents a methylene bridge optionally substituted with phenyl, benzyl, COOH or CO-alkoxy;

Z is CH-R₁₄ or N-R₁₄ wherein R₁₄ represents H or alkyl.

In a more preferred embodiment the substituent pair (R₅/R₆) is identical to the pair (R₇/R₈).

In an even more preferred embodiment either R₅ or R₆ represents H; R₇ and R₈ represents H; R₉ and R₁₀ together represents a methylene bridge and Z is CH₂.

The chiral nitrogen containing organic compound used as catalyst may be chosen among the compounds shown in Table 1a, where the stereoconfiguration shown merely serves an illustrative purpose:

TABLE 1a

| Structure | Name |
|---|---|
| 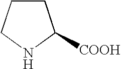 | L-proline |
| 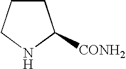 | L-prolinamide |
| 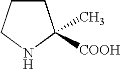 | 2-methyl-L-proline |
| 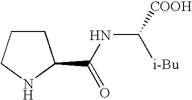 | L-prolyl-L-leucine |
| 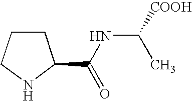 | L-prolyl-L-alanine |
| 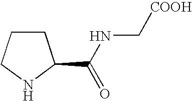 | L-prolylglycine |
| 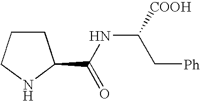 | L-prolyl-L-phenylalanine |
| 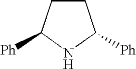 | (2R,5R)-diphenylpyrrolidine |
| 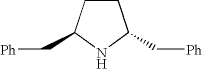 | (2R,5R)-dibenzylpyrrolidine |
| 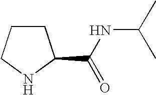 | N-(1-methylethyl)-(2S)-pyrrolidinecarboxamide |
| 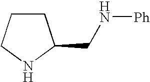 | (2S)-(anilinomethyl)pyrrolidine |
| 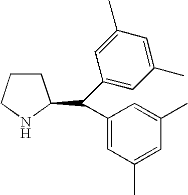 | (2S)-[bis(3,5-dimethylphenyl)methyl]-pyrrolidine |
| 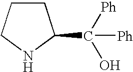 | diphenyl((S)-pyrrolidin-2-yl)methanol |
| 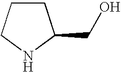 | L-prolinol |
| 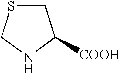 | (4S)-thiazolidinecarboxylic acid |
| 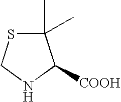 | 5,5-dimethyl-(4S)-thiazolidinecarboxylic acid |
| 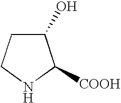 | trans-3-hydroxy-L-proline |
| 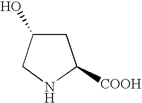 | trans-4-hydroxy-L-proline |
| 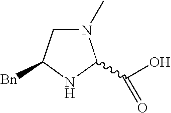 | (4S)-benzyl-1-methyl-imidazolidine-2-carboxylic acid |
| 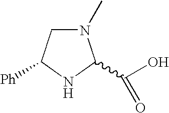 | 1-methyl-(4R)-phenyl-imidazolidine-2-carboxylic acid |
| 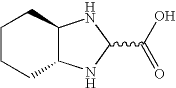 | (4R,5R)-octahydro-benzoimidazole-2-carboxylic acid |
| 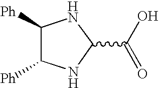 | (4S,5S)-diphenyl-imidazolidine-2-carboxylic acid |
| 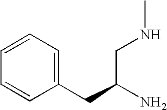 | (S)-N¹-methyl-3-phenyl-propane-1,2-diamine |
| 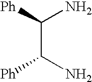 | (1R,2R)-diphenylethanediamine |
| 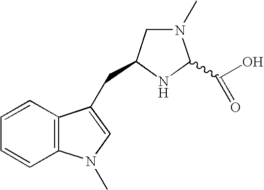 | 1-methyl-(4S)-(1-methyl-1H-indol-3-ylmethyl)- imidazolidine-2-carboxylic acid |
| 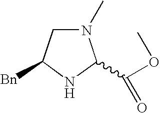 | (4S)-benzyl-1-methyl-imidazolidine-2-carboxylic acid methyl ester |
| 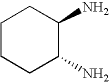 | (1R,2R)-cyclohexanediamine |
| 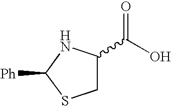 | (2S)-phenyl-thiazolidine-4-carboxylic acid |
| 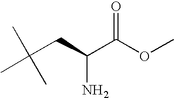 | (S)-tert-leucine methyl ester |
| 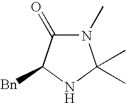 | (5S)-benzyl-2,2,3-trimethyl-imidazolidin-4-one |
| 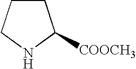 | L-methyl prolinate |
| 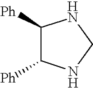 | (R,R)-4,5-diphenylimidazolidine |
| 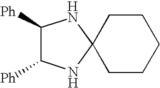 | (R,R)-2-cyclohexyl-4,5-diphenylimidazolidine |

The selection of the stereochemistry of the catalyst depends on the stereochemistry of the desired compound and by proper choice of catalyst one can prepare compounds of either formula (1a) or (1b) as illustrated in the examples. The catalyst can be bound to a support or be unsupported.

The amount of catalyst may be as high as 90 mole percent relative to the compound (2). In principle there is no lower limit to the amount of catalyst employed, however, in practice the desire of a suitable high reaction rate dictates a certain lower limit. The catalyst may conveniently be separated from the final reaction mixture and reused in subsequent reactions according to the present invention.

The reaction may conveniently be carried out at temperatures between −90° C. and 100° C., preferably between −30° C. to 50° C.

No displacement of any other substituents with halogen other than the α-hydrogen atom on the compound (2) is observed in the reaction according to the present invention.

The starting compound (2), and the chiral nitrogen containing organic compounds used as catalysts are commercially available or can be synthesized according to known methods.

Within the general formula (3) are a subclass of novel catalysts of formula (4) which have been found to show a remarkable catalytic effect in asymmetric synthesis of optically active α-halo-carbonyl compounds, in particular α-fluoro-carbonyl compounds, even when applied in amounts less than 5 mol% relative to the compound (2): 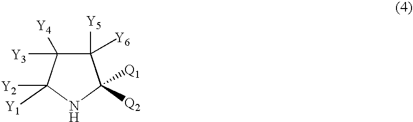
wherein Y₁, Y₂, Y₃, Y₄, Y₅, Y₆ which may be the same or different represents H, an alkyl, haloalkyl, an aryl, an alkylaryl, a heterocycle, a halogen, a hydroxyl, a carbonyl, an alkoxyl, an ester, an amine, an amide, a silyl, a silyl ether, or Y₂ and Y₃ or Y₄ and Y₅ may be bridged together forming part of a ring system one of Q₁ and Q₂ represent H, alkyl, haloalkyl, alkylaryl and the other the group CY₇Y₈(OY₉) wherein Y₇ and Y₈ which may be the same or different represents alkyl, haloalkyl, an alkylaryl, a heterocycle, or optionally substituted aryl and Yg represents a silyl group.

In a preferred embodiment of the present invention Y₁, Y₂, Y₃, Y₄, Y₅, Y₆ each represents H; one of Q₁ and Q₂ represents H; Y₇ and Y₈ each represents an optionally substituted aryl group, wherein the substituents are selected among alkyl and haloalkyl; Yg represents tri-alkyl silyl.

In an even more preferred embodiment Y₁, Y₂, Y₃, Y₄, Y₅, Y₆ each represents H; Y₇ and Y₈ each represents 3,5-di-trifluoromethyl phenyl and Y₉ represents trimethyl silyl.

Illustrative examples of compounds of the formula (4) are shown in Table 1b

TABLE 1b

| Structure | Name |
|---|---|
| 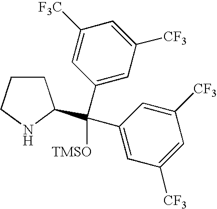 | (S)-2-[bis-(3,5-bis- trifluoromethyl-phenyl)- trimethylsilanyloxy- methyl]-pyrrolidine |
| 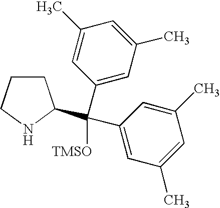 | (S)-2-[bis-(3,5- dimethyl-phenyl)- trimethylsilanyloxy- methyl]-pyrrolidine |
| 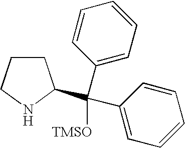 | (S)-2-(diphenyl- trimethylsilanyloxy- methyl)-pyrrolidine |
| 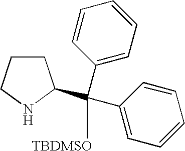 | (S)-2-[(tert-butyl- dimethyl-silanyloxy)- diphenyl- methyl]-pyrrolidine |
| 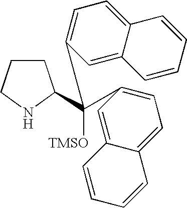 | (S)-2-(di-naphthalen-1- yl-trimethylsilanyloxy- methyl)-pyrrolidine |

The compounds of formula (4) are prepared according to the following reaction scheme: 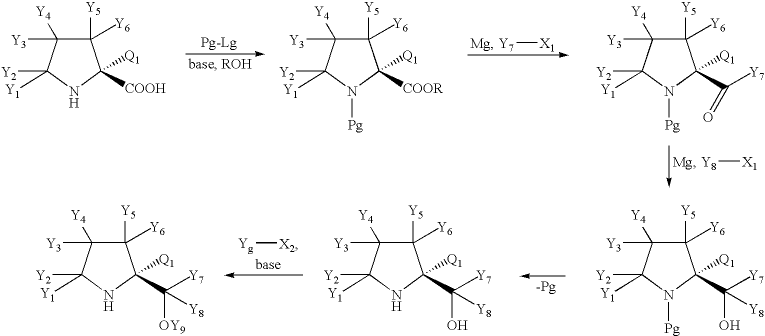
where Y₁, Y₂, Y₃, Y₄, Y₅, Y₆, Y₇, Y₈, Y₉, Q₁ are as previously defined; Pg represents a protecting group such as C(O)O-alkyl; Lg a leaving group such as chloride; X₁ represents e.g. chloro, bromo or iodo and X₂ represents e.g. a halogen or triflate.

The invention is illustrated by the following non-limiting examples:

### EXAMPLE 1

### Preparation of (R)-2-chloro-3-methylbutanal

0.57 g (5.0 mmol) of (L)-prolinamide is added to a stirred solution of 5.4 ml (50 mmol) of 3-methylbutanal in 65 ml of CH₂Cl₂ cooled to 0° C. in an ice bath. 8.7 g (65 mmol) of N-chlorosuccinimide is then added, the ice bath removed and the mixture allowed to warm to 20° C. Stirring is continued until the aldehyde is consumed as shown by ¹H-NMR and gas chromatography (GC) of the mixture after 1-2 h. 200 ml of pentane is then added, and the precipitated solids filtered off. The solvent is then evaporated, and 50 ml of pentane added to the residue. After filtration and evaporation of the pentane (R)-2-chloro-3-methylbutanal was obtained. Yield 5.1 g (85% of theory). The compound is identical to an authentic racemic sample on non-chiral GC and ¹H-NMR. The ee is determined to be 80% by GC on a Chrompack CP-Chirasil Dex CB-column, and the absolute configuration determined as (R) by reduction to 2-chloro-3-methyl-butan-1-ol with NaBH₄ in MeOH and comparison of the optical rotation of this product with the literature value (Koppenhoefer, B.; Weber, R.; Schurig, V. Synthesis 1982, page 317).

### EXAMPLE 2

Using the procedure as in Example 1, the following 2-chlorocarbonyls were obtained:

TABLE 2
Compounds of the formula (1a) or (1b) wherein X is Cl.

| | | | | Yield | Ee |
|---|---|---|---|---|---|
| R | R₂ | R₁ | Catalyst | (%) | (%) |
| Ethyl | H | H | L-prolinamide | 99 | 80(R) |
| Methyl | H | H | ″ | 99 | 75(R) |
| Iso-Propyl | ″ | ″ | ″ | >90 | 87(R) |
| n-Hexyl | ″ | ″ | ″ | 95 | 70(R) |
| Allyl | ″ | ″ | ″ | >90 | 74(nd) |
| Benzyl | ″ | ″ | ″ | 75 | 78(nd) |
| Phenyl | H | CH₃ | ″ | 20 | 16(nd) |
| —(CH₂)₄— | | H | ″ | 30 | 76(nd) |
| Ethyl | H | H | (2R,5R)-diphenyl | >90 | 95(S) |
| | | | pyrrolidine | | |
| Methyl | ″ | ″ | ″ | 99 | 31(nd) |
| Iso-Propyl | ″ | ″ | ″ | >90 | 94(5) |
| tert-Butyl | ″ | ″ | ″ | 30 | 95(nd) |
| n-Hexyl | ″ | ″ | ″ | 99 | 95(S) |
| Allyl | ″ | ″ | ″ | >90 | 95(nd) |
| Benzyl | ″ | ″ | ″ | 82 | 95(nd) |

nd = absolute configuration not determined

### EEXAMPLE

### Preparation of (R)-2-Chloro-3,3-Dimethylbutanal

5.7 mg (0.05 mmol) of (L)-prolinamide is added to a stirred solution of 50 mg (0.5 mmol) of 3,3-dimethylbutanal in 1 ml of CH₂Cl₂ cooled to −78° C. in a dry ice bath. 87 mg (0.65 mmol) of N-chlorosuccinimide is then added, and the mixture is warmed to −24 ° C. Stirring is continued at −24° C. until the aldehyde is consumed as shown by ¹H-NMR and GC of the mixture (approx. 12 h). The yield of (R)-2-chloro-3,3-dimethylbutanal is determined by GC to be >90% of theory. The ee is determined to be 95% by GC on a Chrompack CP-Chirasil Dex CB-column, and the absolute configuration determined as (R) by X-ray crystallography after reduction to (2R)-chloro-3,3 -dimethylbutan-1-ol with NaBH₄.

### EXAMPLE 4

### Preparation of 2-chloro-4-(tert-butyldimethylsilyloxy)-butanal

By the procedure in Example 3, employing 0.10 ml (0.50 mmol) of 4-(tert-butyldimethylsilyloxy)-butanal, (2R)-chloro-4-(tert-butyldimethylsilyloxy)-butanal was obtained. Yield 95% of theory, 81% ee, absolute configuration not determined.

### EXAMPLE 5

### Preparation of enantiomers of 2-chloro-3-methylbutanal

Using the procedure as in Example 1 with 3-methylbutanal, the following results using various catalysts and 1.3 equivalents of N-chlorosuccinimide were obtained:

TABLE 3

| | Cata- | | | | |
|---|---|---|---|---|---|
| | lyst | Reaction | | | |
| | mol | time | | Yield | Ee |
| Catalyst | % | (h) | Solvent | (%) | (%) |
| L-proline | 20 | 1 | CHCl₃ | >95 | 23(R) |
| ″ | 20 | 1 | CH₂Cl₂ | >95 | 25(R) |
| 2-methyl-L-proline | 20 | 5 | DCE | 76 | 60(R) |
| L-prolineamide | 20 | 3 | DCE | >95 | 78(R) |
| ″ | 20 | 1 | EtOH | <5 | 28(R) |
| ″ | 20 | 1 | THF | 23 | 30(R) |
| ″ | 10 | 1 | CH₂Cl₂ | >95 | 82(R) |
| 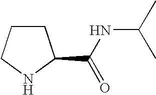 | 20 | 0.5 | DCE | >95 | 54(R) |
| L-prolylglycine | 20 | 1 | DCE | 33 | 81(R) |
| L-prolinol | 20 | 1 | DCE | 34 | 77(R) |
| 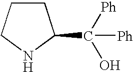 | 20 | 1 | DCE | 15 | 85(R) |
| 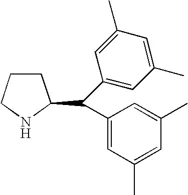 | 20 | 0.5 | DCE | 92 | 64(S) |
| (2R,5R)- | 20 | 0.5 | DCE | >95 | 94(S) |
| diphenylpyrrolidine | | | | | |
| (2R,5R)- | 10 | 1 | DCE | >95 | 94(S) |
| diphenylpyrrolidine | | | | | |
| (2R,5R)- | 5 | 1 | DCE | 77 | 94(S) |
| diphenylpyrrolidine | | | | | |
| (2R,5R)- | 20 | 1 | DCE | <10 | 78(R) |
| dibenzylpyrrolidine | | | | | |
| L-prolyl-L-leucine | 20 | 1 | DCE | 39 | 57(R) |
| L-prolyl-L-phenylalanine | 20 | 1 | DCE | 31 | 59(R) |
| L-prolyl-L-alanine | 20 | 1 | DCE | 21 | 61(R) |
| 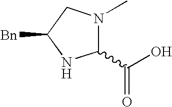 | 20 | 1 | DCE | 52 | 23(S) |
| (1R,2R)- | 10 | 18 | CH₂Cl₂ | 18 | 15(R) |
| cyclohexanediamine | | | | | |
| (1R,2R)- | 10 | 18 | CH₂Cl₂ | 16 | 73(R) |
| diphenylethanediamine | | | | | |

DCE = 1,2-Dichloroethane, THF = Tetrahydrofuran.

### EXAMPLE 6

Using the procedure as in Example 1 with 3-methyl butanal, the following results using different halogenating reagents and 20 mol % of various catalysts:

TABLE 4

| | |
|---|---|
| | (1′) |
| 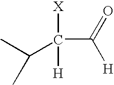 | |

| | Equivalents | | | | |
|---|---|---|---|---|---|
| | relative to | | | Yield | Ee |
| Halogenation agent | compound (2) | Catalyst | Solvent | (%) | (%) |
| 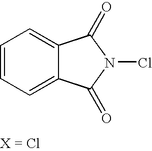 | 2.0 | L-prolinamide | DCE | 17 | 76(R) |
| 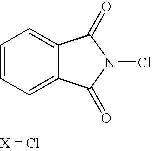 | 2.0 | (2R,5R)- diphenylpyrrolidine | DCE | 26 | 93(S) |
| 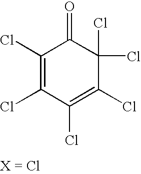 | 1.3 | (2R,5R)- diphenylpyrrolidine | CH₂Cl₂ | 80 | 95(S) |
| 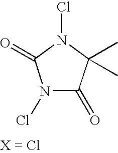 | 1.3 | (2R,5R)- diphenylpyrrolidine | CH₂Cl₂ | 12 | 76(S) |
| 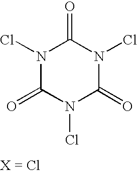 | 1.0 | L-prolinamide | CH₂Cl₂ | 20 | 61(R) |
| 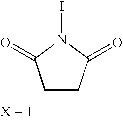 | 2.0 | (2R,5R)- diphenylpyrrolidine | DCE | 100 | 24(nd) |
| 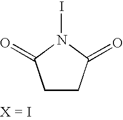 | 2.0 | L-prolinamide | DCE | 22 | 13(nd) |

DCE = 1,2-Dichloroethane.
nd = absolute configuration not determined.

### EXAMPLE 7

### Preparation of 2-bromo-3,3-dimethylbutanal

11.1 mg (0.05 mmol) of (2R,5R)-diphenylpyrrolidine is added to a stirred solution of 50 mg (0.5 mmol) of 3,3-dimethylbutanal in 1 ml of CH₂Cl₂ cooled to −78° C. in a dry ice bath. 115.7 mg (0.65 mmol) of N-bromosuccinimide is then added, and the mixture is warmed to −24° C. Stirring is continued at −24° C. until the aldehyde is consumed as shown by ¹H-NMR and GC of the mixture (approx. 2 h). The yield of 2-bromo-3,3-dimethylbutanal is determined by GC to be ca. 10% of theory. The ee is determined to be 80% by GC on a Chrompack CP-Chirasil Dex CB-column, absolute configuration not determined.

### EXAMPLE 8

### Preparation of 2-chlorocyclohexanone

A series of experiments were performed to prepare optically active 2-chlorocyclohexanone from cyclohexanone in the presence of various catalysts using the following procedure: To a mixture of cyclohexanone and catalyst in CH₂Cl₂ was added N-chlorosuccinimide (0.5 nmiol) and the reaction mixture stirred at ambient temperature for the time indicated in Table 5. Ee was determined by CSP-GC and the yield determined by GC.

TABLE 5

| | | Cata- | | | |
|---|---|---|---|---|---|
| | Cyclo- | lyst | Reaction | | |
| | hexanone | mol | time | Yield | Ee |
| Catalyst | (mmol) | % | (h) | (%) | (%) |
| L-prolinamide | 2.5 | 20 | 24* | 40 | 81(R) |
| L-methyl prolinate | 2.5 | 20 | 24 | 20 | 20(R) |
| 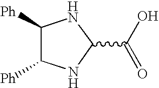 | 2.5 | 20 | 0.75 | 10 | 62(R) |
| 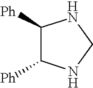 | 2.5 | 20 | 20** | 88 | 95(R) |
| 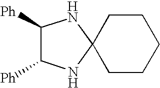 | 2.5 | 20 | 22 | 17 | 88(R) |

*Reaction performed at −24° C.
**Reaction performed at −10° C.

### EXAMPLE 9

### Influence of Addition of Organic Acids

A series of experiments were performed to prepare optically active 3-chlorotetrahydropyran-4-one from tetrahydropyran-4-one, in various solvents using (R,R)-4,5-diphenylimidazolidine as catalyst and in the presence of an organic acid, by the following procedure: To a mixture of tetrahydropyran-4-one, organic acid (0.4 molar equivalent), solvent (1 mL), and the catalyst (0.05 mmol), was added N-chlorosuccinimide and the reaction mixture stirred at −10° C. for a period of 24 h. Ee was determined by CSP-GC and the yield determined by GC.

TABLE 6

| Tetrahydro- | | | | | |
|---|---|---|---|---|---|
| pyran-4-one | | | NCS | Yield | Ee |
| (mmol) | Acid | Solvent | (Equiv.) | (%) | (%) |
| 5 | — | CH₂Cl₂ | 1 | 30 | 30 |
| 5 | PhCO₂H | CH₂Cl₂ | 1 | 53 | 84 |
| 2.5 | PhCO₂H | MeCN | 1 | 15 | 97 |
| 2.5 | AcOH | MeCN | 1 | 19 | 87 |
| 5 | CF₃CO₂H | CH₂Cl₂ | 1 | 62 | 68 |
| 2.5 | ClCH₂CO₂H | MeCN | 1 | 50 | 91 |
| 1 | 2-NO₂—PhCO₂H | MeCN | 1.5 | 63 | 97 |
| 1 | 2-NO₂—PhCO₂H | MeCN | 2.0 | 72 | 98 |

### EXAMPLE 10

### Preparation of α-halo Cyclic and Acyclic Ketones

A series of experiments were performed to prepare optically active α-halo cyclic and acyclic ketones from the corresponding ketone using the following general procedure: To mixture of ketone, (R,R)-4,5-diphenylimidazolidine as catalyst and 2-NO₂-PhCO₂H in MeCN was added N-chlorosuccinimide (1.0 mmol) and the reaction stirred for a period of 20 h. Ee was determined by CSP-GC and the yield determined by ¹H NMR using an internal standard and confirmed using GC analysis.

TABLE 7

| | | | Reaction | | |
|---|---|---|---|---|---|
| Ketone | 2-NO₂—PhCO₂H | Catalyst | temp | Yield | Ee |
| (mmol) | (mmol) | (mmol) | (° C.) | (%) | (%) |
| 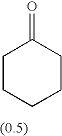 | 0.25 | 0.1 | −24 | 82 | 97 |
| 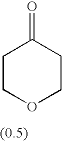 | 0.125 | 0.05 | −24 | 72 | 98 |
| 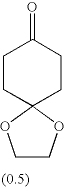 | 0.25 | 0.1 | −24 | 83 | 90 |
| 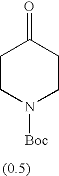 | 0.25 | 0.1 | −24 | 76 | 93 |
| 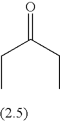 | 0.25 | 0.1 | −10 | 62 | 83 |
| 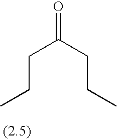 | 0.25 | 0.1 | −10 | 40 | 88 |

### EXAMPLE 11

### Preparation of α-bromo cyclohexanone

A series of experiments were performed to prepare α-bromo cyclohexanone:

TABLE 8

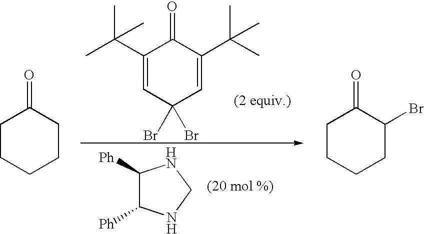

| | | | | Yield | Ee |
|---|---|---|---|---|---|
| Acid (mol %) | Temp (° C.) | Solvent | Time (h) | (%) | (%) |
| 2-NO₂—PhCO₂H (40) | −10 | MeCN | 3.5 | 30 | 83 |
| 2-NO₂—PhCO₂H (40) | −24 | MeCN | 20 | 32 | 82 |
| 2-NO₂—PhCO₂H (40) | −10 | Et₂O | 20 | 86 | 80 |
| 2-NO₂—PhCO₂H (40) | −10 | Et₂O | 2.5 | 65 | 88 |
| None | −10 | CH₂Cl₂ | 1 | 5 | >99 |
| 2-NO₂—PhCO₂H (40) | −10 | Toluene | 3 | 25 | 90 |
| 2-NO₂—PhCO₂H (40) | −10 | Toluene | 20 | 60 | 82 |
| 2-NO₂—PhCO₂H (40) | −10 | Acetone | 5 | 57 | 88 |
| AcOH (40) | −10 | CH₂Cl₂ | 2 | 47 | 89 |
| AcOH (40) | −10 | CH₂Cl₂ | 20 | 52 | 86 |
| PhCO₂H (40) | −10 | CH₂Cl₂ | 20 | 79 | 83 |
| PhCO₂H (40) | −24 | CH₂Cl₂ | 4.5 | 65 | 86 |
| PhCO₂H (40) | −24 | Et₂O | 20 | 60 | 89 |

### EXAMPLE 12

### Preparation of α-bromo tetrahydropyran-4-one

A series of experiments were performed to prepare α-bromo tetrahydropyran-4-one:

TABLE 9

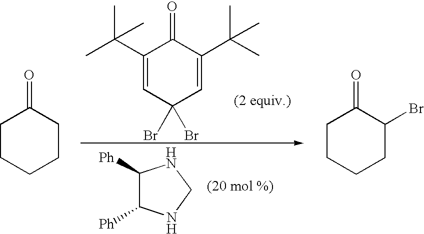

| | | | | | Ee |
|---|---|---|---|---|---|
| Acid (mol %) | Temp (° C.) | Solvent | Time (h) | Yield (%) | (%) |
| PhCO₂H (40) | −30 | THF | 20 | 66 | 88 |
| PhCO₂H (40) | −30 | THF | 40 | 82 | 85 |
| PhCO₂H (40) | −30 | t-BuOCH₃ | 40 | 61 | 86 |
| PhCO₂H (40) | −30 | THF | 40 | 97 | 89 |

### EXAMPLE 13

### Preparation of α-fluoro-3,3-dimethylbutanal

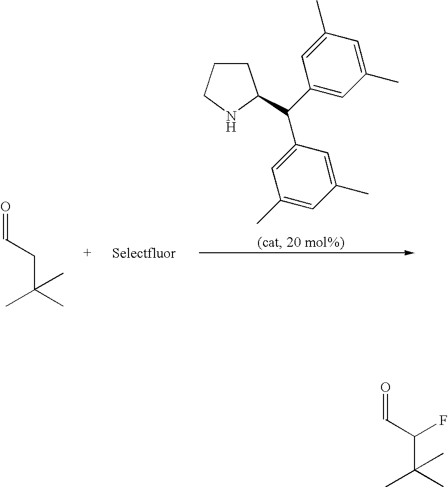

The catalyst (0.1 mmol) and 3,3-dimethyl-butyraldehyde (0.5 mmol) are stirred in CH₃CN (1.0 mL) for 30 min at room temperature. Selectfluor (106 mg, 0.60 mmol, 1.2 eq.) is added and the reaction mixture is stirred for 20 h. GC analysis showes 65% conversion of the aldehyde and 71% ee for the α-fluoro-3,3-dimethylbutanal. Selectfluor is a trademark of Air Products, and the compound name is 1-chloromethyl-4-fluoro-1,4-diazoniabicyclo[2.2.2]octane bis(tetrafluoroborate).

### EXAMPLE 14

### Preparation of α-fluoro aldehydes

A series of experiments were performed using different aldehydes, fluorinating agents and catalysts at room temperature:

TABLE 10

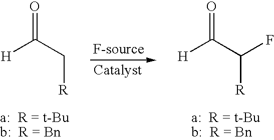

| Aldehyde | Catalyst (mol %) | Fluor source | Solvent | Time (h) | Conversion (%) | Ee (%) |
|---|---|---|---|---|---|---|
| 1a | 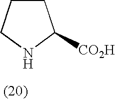 | Selectfluor | MeCN | 20 | 88 | 34 |
| 1a | 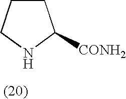 | Selectfluor | MeCN | 20 | 98 | 24 |
| 1a | 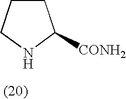 | NFSI | MeCN | 1 | 38 | 27 |
| 1a | 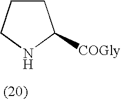 | Selectfluor | MeCN | 20 | 98 | 45 |
| 1a | 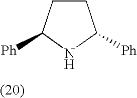 | Selectfluor | MeCN | 1 | 24 | 78 |
| 1a | 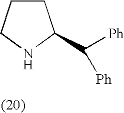 | Selectfluor | MeCN | 20 | 63 | 71 |
| 1a | 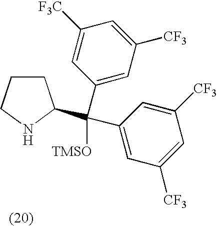 | NFSI | MeCN | 20 | 90 | 94 |
| 1a | 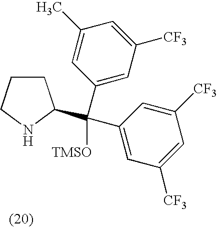 | NFSI | MeCN | 20 | 36 | 95 |
| 1b | 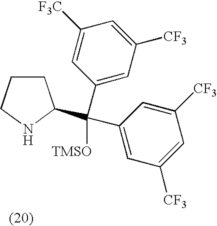 | NFSI | MeCN | 20 | 45 | 95 |
| 1b | 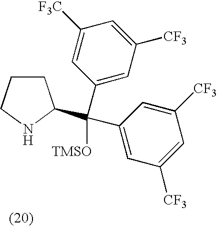 | NFSI | CH₂Cl₂ | 20 | 58 | 97 |
| 1b | 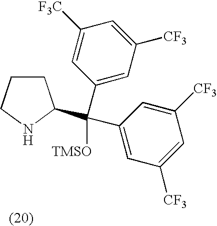 | NFSI | MTBE | 1 | 96 | 93 |
| 1b | 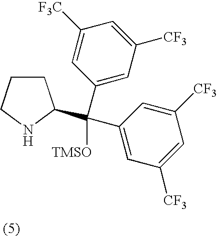 | NFSI | MTBE | 1 | 77 | 96 |
| 1b | 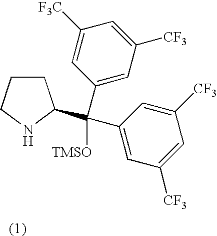 | NFSI | MTBE | 2 | 92 | 93 |
| 1a | 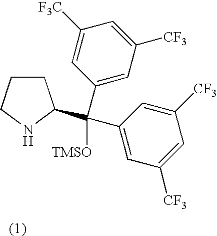 | NFSI | MTBE | 2 | >95 | 97 |

### EEXAMPLE 15

### Procedure for the organocatalytic α-fluorination of aldehydes using NFSI as the Fluorinating Agent Catalyzed by ((S)-2-[bis-(3-5-bistrifluoromethyl-phenyl)-trimethylsilanyloxy-methyl]-pyrrolidine.

The catalyst ((S)-2-[bis-(3-5-bistrifluoromethyl-phenyl)-trimethylsilanyloxy-methyl]-pyrrolidine, 0.005 mmol, 1 mol %) and the aldehyde (0.75 imnol, 1.5 eq.) are stirred in MTBE (1.0 ml) for 30 min at room temperature. NFSI (158 mg, 0.50 mnol, 1.0 eq.) is added and the reaction mixture is stirred for 2 h at room temperature. Conversion is determined by GC analysis. The yields are also confirmed after reduction of the catalytic product to the 10 corresponding alcohol by the following procedure: Pentane (4.0 ml) is added and the precipitates are removed by filtration. MeOH (4.0 ml) is added followed by NaBH₄ (2 eq). The reaction is quenched after 1 h with a 1M solution of KHSO₄ and the product is extracted with Et₂O. The organic phase is dried on Na₂SO₄, filtrated and after evaporation of the solvent the alcohol is isolated by flash chromatography on silica.

TABLE 11

| Aldehyde | Yield (%) | Ee (%) |
|---|---|---|
| 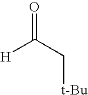 | >90 | 97 |
| 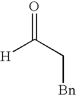 | 74 | 93 |
| 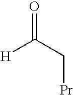 | 74 | 96 |
| 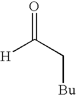 | 74 | 96 |
| 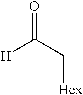 | 55 | 96 |
| 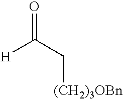 | 64 | 91 |
| 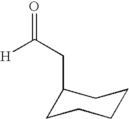 | 60 | 96 |
| 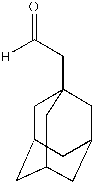 | 75 | 96 |
| 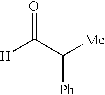 | 70 | 53 |

### EXAMPLE 16

### Preparation of the catalyst (S)-2-[bis-(3-5-bistrifluoromethyl-phenyl)-trimethylsilanyloxy-methyl]-pyrrolidine.

The catalyst ((S)-2-[bis-(3-5-bistrifluoromethyl-phenyl)-trimethylsilanyloxy-methyl]-pyrrolidine is prepared by a four steps synthesis from L-proline. The detailed procedures are the following:

1. Preparation of (S)-pyrrolidine-1,2-dicarboxyclic acid 1-ethyl ester 2-methyl ester: 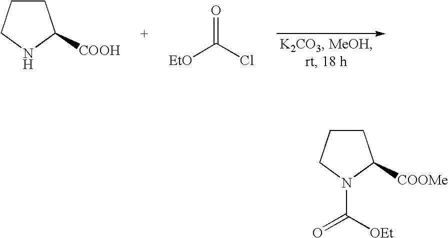
45 ml (477 nmuol) of ethyl chloroformate is added to a stirred suspension of 25 g (217 mmol) L-proline and 30 g (217 mmol) potassium carbonate in 300 ml MeOH. The reaction is stirred at ambient temperature overnight. Evaporation of the solvent, addition of 200 ml water, extraction with CH₂Cl₂ (4×100 ml), drying of the organic phase over Na₂SO₄ and removal of the solvent yield 44 g (99%) of the pure product.

2. Preparation of (S)-1,2-bis-(3,5-bis-trifluoromethyl-phenyl)-tetrahydro-pyrrolo[1,2-c]oxazol-3-one:

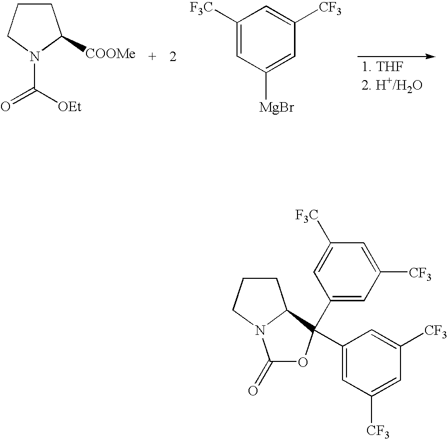

0.84 g (34 mmol) of Mg is suspended in 20 ml of dry THF under a N₂ atmosphere and a solution of 5.9 ml (34 mmol) of 2,5-bis(trifluoromethyl)bromobenzene in 60 ml of dry TUF is added slowly. Afterwards the mixture is heated up to reflux for 1 h. The reaction is cooled down to 0° C. and a solution of 3.11 g (15 mmol) pyrrolidine-1,2-dicarboxyclic acid 1-ethyl ester 2-methyl ester in 50 ml of dry THF is added. Then the reaction is allowed to reach room temperature before refluxing for 2 h. The reaction mixture is cooled down to room temperature and then poured into a mixture of ice and saturated NH₄Cl solution. Extraction with EtOAc (3×50 ml), drying over Na₂SO₄ and evaporation of the solvent yield 49.0 g (99%) of a dark brown solid/oil. Recrystallisation from Et₂O yield 4.3 g (50%) of the product as a white solid.

3. Preparation of (S)-bis-(3,5-bis-trifluoromethyl-phenyl)-pyrrolidin-2-yl-methanol:

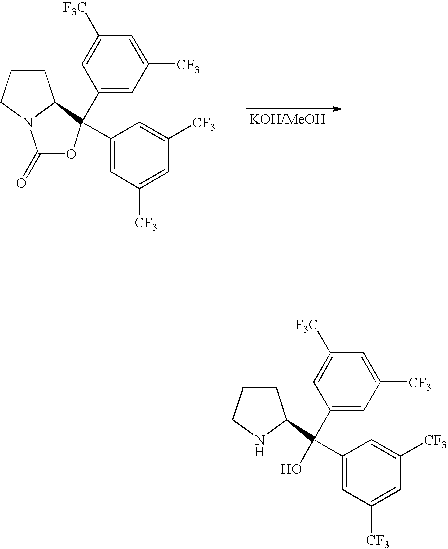

4.3 g (76 mmol) KOH and 4.2 g (7.6 mmol) (S)-1,2-bis-(3,5-bis-trifluoromethyl-phenyl)-tetrahydro-pyrrolo[1,2-c]oxazol-3-one are suspended in 20 ml MeOH and heated up to reflux for 2 h. After reaching ambient temperature and removal of the solvent water is added and the mixture is extracted with CH₂Cl₂. Drying over Na₂SO₄ and evaporation yield 4.2 g (99%) of the product as a colorless oil.

4. Preparation of (S)-2-[bis-(3-5-bistrifluoromethyl-phenyl)-trimethylsilanyloxy-methyl]-pyrrolidine

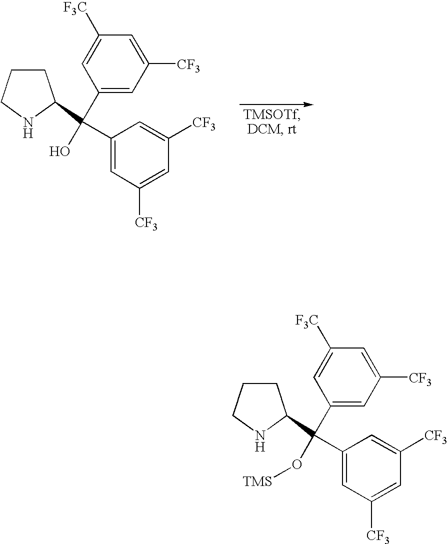

2.0 ml (11.4 mmol) TMSOTf is added at 0° C. to a solution of 4.0 g (7.6 mmol) (S)-bis-(3,5-bis-trifluoromethyl-phenyl)-pyrrolidin-2-yl-methanol and 1.59 ml (11.4 mmol) Et₃N in 50 ml CH₂Cl₂. The reaction is then allowed to reach ambient temperature and stirred for 1 h until full conversion of the starting material is confirmed by TLC analysis. The reaction is quenched with water, the product extracted with CH₂Cl₂ (3×30 ml) and dried over Na₂SO₄. After evaporation of the solvent the product was purified by flash chromatography on silica (pentane:CH₂Cl₂=2:1) to yield 3.8 g (84%) of the catalyst as a yellow oil, which after precipitation affords a colorless solid.

1. A process for the catalytic asymmetric synthesis of an optically active compound of the formula (1a) or (1b) 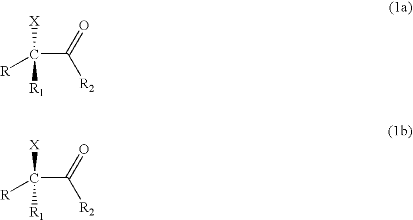
wherein R is an organic group; X is halogen; R₁ and R₂ which may be the same or different represents H, or an organic group or R₁ and R₂ may be bridged together forming part of a ring system; R and R₂ may be bridged together forming part of a ring system; with the provisio that R and R₁ are different and R₂ when different from H is attached through a carbon-carbon bond, comprising the step of reacting a compound of the formula (2) 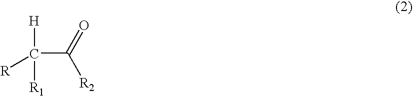
with a halogenating agent in the presence of a catalytic amount of a chiral nitrogen containing organic compound.

2. The process according to claim 1, wherein R₂ is H or an optionally substituted C₁₋₁₀ alkyl group or R and R₂ are bridged together forming part of a ring system.

3. The process according to claim 1, wherein R₁ is H or an optionally substituted C₁₋₁₀ alkyl group.

4. The process according to claim 1, wherein R is an optionally substituted C₁₋₁₀ alkyl group, an optionally substituted C₂₋₈ alkylene group or a C₁₋₃-alkylaryl group.

5. The process according to claim 4 wherein R is an optionally substituted C₁₋₆ alkyl group, an optionally substituted C₂₋₄ alkylene group or a C₁₋₂-alkylaryl group.

6. The process according to claim 4 wherein R₁ and R₂ are H.

7. The process according to claim 1 wherein the chiral nitrogen containing organic compound is selected among compounds having a primary or secondary nitrogen atom or when appropriate in one of its salt forms.

8. The process according to claim 7 wherein the chiral nitrogen containing organic compound is selected among compounds of the formula (3) 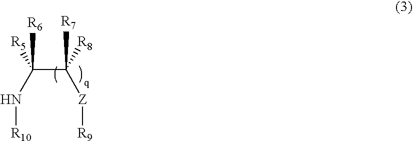
wherein q is 0 or 1;
R₅, R₆, R₇, R₈, which may be the same or different represents H, alkyl, haloalkyl, alkoxyl, OH, amino, amide, silyl, silyl ether, COR₁₁, optionally substituted aryl, an optionally substituted heterocycle, alkyl substituted with at least one OH group, an optionally substituted amino group or optionally substituted aryl or heterocycle or R₅ and R₆ together or R₇ and R₈ together may represent a carbonyl group or when q is 1, R₅ with either R₇ or R₈ may be bridged together forming part of a ring system; R₁₁ represents an optionally substituted amino group or OR₁₂ wherein R₁₂ represents H, alkyl or phenyl;
R₉ and R₁₀, which may the same or different represents H, alkyl, OH, or alkoxy; or R₉ and R₁₀ may be bridged together forming part of a ring system;
Z is S, O, C═O, C(R₁₄)₂, N—R₁₄ wherein R₁₄ is R₅;
with the provisio that the groups R₅, R₆, R₇, R₈, R₉, R₁₀, R₁₄, and Z are selected so that the compound (3) is a chiral compound.

9. The process according to claim 8 wherein q is 1; R₅, R₆, R₇, R₈ which may the same or different represents H, COR₁₁, optionally substituted aryl or methyl substituted with at least one of the following, an OH group, an optionally substituted amino group or an optionally substituted aryl or heterocycle group; or R₅ and R₇ together represents a C₃₋₅ alkylene bridge;
R₁₁ represents OH, NH₂ or NH-alkyl;
R₉ and R₁₀ are H or R₉ and R₁₀ together represents a methylene bridge optionally substituted with phenyl, benzyl, COOH or CO-alkoxy;
Z is CH—R₁₄ or N—R₁₄ wherein R₁₄ represents H or alkyl.

10. The process according to claim 9 wherein either R₅ or R₆ represents H; R₇ and R₈ represents H; R₉ and R₁₀ together represents a methylene bridge and Z is CH₂.

11. The process according to claim 3 wherein R₁ is H and R and R₂ each represents an optionally substituted C₁₋₁₀ alkyl group or R₂ together with R forms an optionally substituted C₃₋₅-alkylene bridge optionally with one or more of the carbon atoms being replaced by a heteroatom.

12. The process according to claim 1 wherein one or more acids are added to the reaction media.

13. The process according to claim 8, wherein the compound of formula (3) is a compound of formula (4) 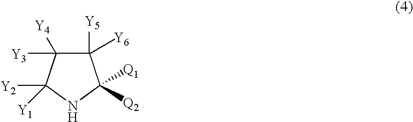
wherein Y₁, Y₂, Y₃, Y₄, Y₅, Y₆ which may be the same or different represents H, an alkyl, haloalkyl, an aryl, an alkylaryl, a heterocycle, a halogen, a hydroxyl, a carbonyl, an alkoxyl, an ester, an amine, an amide, a silyl, a silyl ether, or Y₂ and Y₃ or Y₄ and Y₅ may be bridged together forming part of a ring system one of Q₁ and Q2 represent H, alkyl, haloalkyl, alkylaryl and the other the group CY₇Y₈(OY₉) wherein Y7 and Y₈ which may be the same or different represents alkyl, haloalkyl, an alkylaryl, a heterocycle, or optionally substituted aryl and Y₉ represents a silyl group.

14. A compound of the formula (4) as disclosed in claim 13.

15. The compound according to claim 14, wherein Y₁, Y₂, Y₃, Y₄, Y₅, Y₆ each represents H; one of Q₁ and Q₂ repre;sents H; Y₇ and Y₈ each represents an optionally substituted aryl group, wherein the substituents are selected among alkyl and haloalkyl; Y₉ represents tri-alkyl silyl.

16. The compound according to claim 15, wherein Y₇ and Y₈ each represents 3,5-di-trifluoromethyl phenyl and Y₉ represents trimethyl silyl.

17. The compound according to claim 15, wherein Y₇ and Y₈ each represents 3,5-di-methyl phenyl and Y₉ represents trimethyl silyl.
